(19) United States
(12) Patent Application Publication
(10) Pub. No.: US 2010/0297198 A1
(43) Pub. Date: Nov. 25, 2010

(54) LIQUID-TYPE DENTIFRICE COMPOSITION CONTAINED SILVER PARTICLES AND MOUSSE-TYPE DENTIFRICE USING THE SAME

(76) Inventor: Ki-Young Kim, Seoul (KR)

Correspondence Address: Jae Y. Park, Kile, Park, Goekjian, Reed & McManus, PLLC, 1200 New Hampshire Ave. NW, Suite 570, Washington, DC, 20036, US

(21) Appl. No.: 12/517,837

(22) PCT Filed: Dec. 8, 2007
(86) PCT No.: PCT/KR2007/006384

§ 371 Date: Jun. 5, 2009

(30) Foreign Application Priority Data
Dec. 8, 2006 (KR) 10-2006-0124431

(51) Int. Cl. A61K 9/14; A61K 8/46; A61Q 11/00

(52) U.S. Cl. 424/401; 424/56; 424/52; 424/45

(57) ABSTRACT

Disclosed are a liquid type dentifrice composition primarily containing silver particles which is drawn out in foam state when used to brush teeth to prevent the liquid dentifrice from flowing down during tooth-brushing, resulting in effective and convenient tooth-brushing function and exhibit bactericidal action and protect mouth and teeth from periodontal disease and other mouth diseases because of original properties of silver ingredients in the dentifrice composition; and, in addition to, a mousse type dentifrice formulation using the same that improves aesthetic property and product quality thereof. The inventive dentifrice composition primarily containing silver particles useful for mouthwash comprises: at least one surfactant selected from a group consisting of 0.5 to 3% by weight of SQdium lauryl sulfate, 0.05 to 1% by weight of sodium sarcosine laurate, 0.5 to 5% by weight of alkyl polyglucoside and 0.5 to 3% by weight of sucrose fatty acid ester; at least one wetting agent selected from a group consisting of 30 to 80% by weight of sorbitol solution and 0.5 to 5% by weight of glycerine; at least one solubilizing agent selected from a group consisting of 0.1 to 1% by weight of hydrogenated polyoxyethylene caster oil and 0.1 to 1% by weight of polyoxyethylene sorbitan monolaurate effective to solubilize flavor and active ingredients; 0.01 to 10% by weight of silver particles with bactericidal action; and the balance of purified water relative to total weight of the composition. The inventive mousse type dentifrice formulation comprising the above liquid dentifrice composition is prepared by steps of: introducing the dentifrice composition comprising silver particles as essential ingredient into a foam generating receptacle with constant capacity; and drawing the composition in foam form through outlet of the receptacle in order to brush teeth.

### TECHNICAL FIELD

The present invention relates to liquid type dentifrice compositions primarily containing silver particles and mousse type dentifrices using the same, more particularly, a liquid type dentifrice composition containing silver particles as essential component which is normally retained in liquid form in a receptacle body containing the dentifrice but drawn out in foam state by squeezing the receptacle body when used to brush teeth, so that it prevents the dentifrice from flowing down during tooth-brushing and, in addition to, a mousse type dentifrice formulation using the same.

### BACKGROUND ART

Conventional dentifrice products used for tooth-brushing and rinsing are commercially available and distributed in different types of formulation such as, for example, paste, powder, gel and viscous liquid, liquid, etc. and have various advantages and disadvantages in handling and use thereof. For example, a dentifrice composition in paste form has high viscosity, is apt to cause damage of tooth enamel layer due to polishing ingredient contained in the composition, is hard to be drawn out by squeezing a receptacle containing the composition as well as practical difficulty in completely using content of the receptacle, whereby it induces disposal of the used receptacle containing residual content thereof and then environmental pollution. In case of a powdery dentifrice composition, it shows inconvenience in use since, particles of the dentifrice scatter and are sprayed out. For a gel type dentifrice composition, it is classified into ones with and without polishing agent that have high viscosity and less dispersion property to cause poor dispersion of dentifrice ingredient of the composition in mouth of a user, thereby having inconvenience in using the composition. Furthermore, a dentifrice composition in liquid form has a disadvantage of easily flowing down due to low viscosity, although it is widely used at present as mouthwash or breath freshener that inhibits Streptococcus mutans and/or removes bad breath. Since the liquid composition lacks content of cleaning ingredients and has problems of low viscosity and easily flowing down, it does not represent tooth-brushing effect sufficient to remove dental plaque or Streptococcus mutans.

One of prior arts in relation to liquid type dentifrice compositions is a liquid cleaner gel disclosed in Korean Patent Application No. 2001-7004081 entitled “Transparent liquid type tooth cleaner gel”, which is transparent at low temperature and moderate in liquid-like fluidity by comprising water and wetting agent in desired relative ratio thereof. In addition, this gel is neither quickly nor deeply settled down to bottom of a tooth brush when the gel is applied on brushing face of the tooth brush. However, such cleaner gel exhibits less shape retention (if a material has higher shape retention it shows substantially no fluidity, whereas the material flows like water in case of the shape retention being lowered) and low fluidity. Therefore, the cleaner gel has inconvenience in application of tooth cleaners.

Therefore, in order to solve the above problems, Korean Patent Application No. 2002-0053430 which is entitled “Liquid type dentifrice composition,” proposed that shape retention and dispersion stability of a low viscosity liquid dentifrice can be enhanced by adding sodium caseinate to prepared liquid dentifrice material.

Conventional liquid dentifrice compositions, more particularly, the above disclosed composition has constant level of shape retention and dispersion stability sufficient to ensure function of liquid dentifrice.

However, the above known liquid composition cannot completely solve the problem of easily flowing down due to low viscosity during tooth-brushing, thus, be difficult to brush teeth. Further, the known composition required any chemical additive to assist dental cleaning and/or bactericidal effect to be added, which potentially causes stimulation against sensitive oral mucosa or other adverse effect. Also, when the liquid dentifrice is formed in mere liquid, the composition has problems such as poor effect which is not enough to attract customer's interest and lowered product quality thereof.

Alternatively, fluorine based formulations which are widely used to give bactericidal action in generally known toothpastes, efficiently carried out to inhibit bacteria and/or protect cavity. However, for gingiva for supporting tooth and/or inner skin of mouth, the fluorine based formulations demonstrated relatively lowered bactericidal effect and had problems such as generation of spots on tooth or displeasure caused by irritant odor of chemicals.

Accordingly, in order to solve problems caused by excessive use of the fluorine based formulations in dentifrice and efficiently eliminate bacteria in oral mucosa or sensitive inner skin of the mouth and/or gingiva, extensive studies and investigation for application of particular materials such as jade, chitosan, natural salt, etc. are now in progressing. But these materials make liquid dentifrice become muddy or opaque, deteriorate quality of the dentifrice and have problems such as, for example: functional deterioration of preservative and surfactant normally contained in the liquid dentifrice; and, when the natural salt is used, lowered effect for removing bad breath and substantially impossible ability to satisfy tastes of all customers due to original salty property of the natural salt, although proliferation of microorganisms can be inhibited by the natural salt.

### DISCLOSURE OF THE INVENTION

### Technical Problem

Accordingly, the present invention is directed to solve the conventional problems described above and, an object of the present invention is to provide a liquid type dentifrice composition containing silver particles as essential ingredient for mouth cleaning, which is drawn out in foam state when used to brush teeth so as to prevent dentifrice liquid from flowing down during tooth-brushing and enhance effect of the tooth-brushing, so that the dentifrice composition can efficiently remove bacteria in mouth and protect mouth and teeth from periodontal disease and other mouth diseases because of original properties of silver ingredients in the dentifrice composition, and a mousse type dentifrice formulation prepared by using the same that improves aesthetic property and, in turn, product quality thereof.

### Technical Means to Solve the Problem

In order to accomplish the above described object, the present invention provides a liquid type dentifrice composition primarily containing silver particles useful for mouthwash, which comprises: at least one surfactant selected from a group consisting of 0.5 to 3% by weight of sodium lauryl sulfate, 0.05 to 1% by weight of sodium sarcosine laurate, 0.5 to 5% by weight of alkyl polyglucoside and 0.5 to 3% by weight of sucrose fatty acid ester; at least one wetting agent selected from a group consisting of 30 to 80% by weight of sorbitol solution and 0.5 to 5% by weight of glycerine; at least one solubilizing agent selected from a group consisting of 0.1 to 1% by weight of hydrogenated polyoxyethylene caster oil and 0.1 to 1% by weight of polyoxyethylene sorbitan monolaurate effective to solubilize flavor and active ingredients; 0.01 to 10% by weight of silver particles with bactericidal action; and the balance of purified water relative to total weight of the composition.

Silver particle moiety in the present inventive dentifrice composition comprises 0.01 to 0.1% by weight of silver foil having particle diameter of 1 to 200 μm.

The present dentifrice composition further includes: solvent containing 1 to 12% by weight of ethanol; 0.5 to 0.2% by weight of at least one flavor ingredient selected from a group consisting of peppermint oil, herb oil, eucalyptus oil, spearmint oil, orange oil and other composite flavor component as food additive; at least one flavoring agent selected from a group consisting of 0.05 to 0.3% by weight of L-menthol and 0.005 to 0.1% by weight of DL-camphor; and 0.05 to 0.15% by weight of sodium saccharin.

Such prepared dentifrice composition according to the present invention can also include active ingredient for protecting gingival disease comprising at least one selected from a group consisting of 0.01 to 0.5% by weight of tocopherol acetate, 0.01 to 0.3% by weight of allantoin and 0.01 to 0.5% by weight of allantoin chlorohydroxyaluminum.

The dentifrice composition can include active ingredient for protecting cavity comprising at least one selected from 0.02 to 0.22% by weight of sodium fluoride and 0.2 to 0.76% by weight of sodium monofluorophosphate, as well as preservative containing 0.01 to 0.2% by weight of methyl p-hydroxybenzoate.

According to features of the present invention to accomplish the above object, the present invention provides a mousse type dentifrice formulation comprising the liquid dentifrice composition according to the present invention, which is prepared by introducing the dentifrice composition comprising silver particles as essential ingredient into a foam generating receptacle with constant capacity and drawing the composition in foam form through outlet of the receptacle in order to brush teeth.

The receptacle of the dentifrice formulation is a pump type foam generating receptacle consisting of: a main body with constant capacity to contain the dentifrice composition; a pump device that is arranged on a fixture cap coupled to output hole of the main body and has an inhalation nozzle which is got into the main body to suck content of the main body and an output nozzle through which the suck dentifrice composition is discharged out of the receptacle.

The receptacle may adopt an aerosol form of receptacle with a construction of mixing the dentifrice composition with gas and outputting the mixture by pressing a button of the receptacle or alternative construction consisting of an inner container for receiving the dentifrice composition and an outer container to house the inner container inside while being filled with gas around outside of the inner container.

The gas filled in the aerosol form of receptacle includes a mixture of propane gas and butane gas in mixing ratio of 30:70, liquefied petroleum gas with 100% butane gas or compressed air.

The liquid type dentifrice composition primarily containing silver particles and the mousse type dentifrice prepared by using the same according to the present invention will be described in detail based on techniques for producing the liquid dentifrice. For the interest of clarity, general description for construction, functional behavior and effect of the dentifrice composition and formulations which are easily known by persons skilled in the state of prior art should be eliminated or simplified while only essential parts of the invention being understood in the present detailed description.

In a preferred embodiment of the present invention, a liquid type dentifrice composition primarily containing silver particles comprises: anion surfactant and non-ionic surfactant for generating foam; wetting agent for preventing dentifrice ingredient from being cured or converted into solid state during exposure in air; solubilizing agent to solubilize active ingredients of the composition; silver particles with bactericidal action to eliminate bacteria in mouth and teeth; and purified water.

The anion surfactant contained in the dentifrice composition includes at least one or two selected from 0.5 to 3 wt. % of sodium lauryl sulfate and 0.05 to 1 wt. % of sodium sarcosine laurate. The non-ionic surfactant includes at least one selected from 0.5 to 5 wt. % of alkylpolyglucoside and 0.5 to 3 wt. % of sucrose fatty acid ester.

The wetting agent includes at least one or two selected from 30 to 80 wt. % of sorbitol solution and 0.5 to 5 wt. % of glycerine in order to improve flavor taste in mouth and prevent or reduce dry mouth.

The solubilizing agent includes at least one selected from 0.1 to 1 wt. % of hydrogenated polyoxyethylene caster oil and 0.1 to 1 wt. % of polyoxyethylene sorbitan monolaurate in order to solubilize flavor, effective ingredients and active ingredients of the composition.

Content of silver particles contained in the dentifrice composition ranges from 0.01 to 10 wt. %, preferably 0.01 to 0.1 wt. % relative to total weight of the composition and the silver particles are formed by preparing silver foil with particle size of 1 to 200 μm from silver component having 99.9% purity, and introducing and agitating the silver foil in a container to have homogeneous distribution thereof.

It is well known that silver has excellent sterilization and deodorization properties, which easily penetrates into skin or tissues of a human being and serves as a catalyst to stop function of enzymes required in oxygen metabolism by bacteria, fungi or virus and to derive apotosis thereof in a short period of time so that it is useful for treating and protecting all kinds of diseases caused by various bacteria, virus and/or fungi. Although silver is traditionally used in oriental medical application including, for example, additives or medical device, a number of investigations and studies are now in progress to use silver in more extended applications. Silver functions as a natural bactericide without adverse effect, and activates physiological function of human skin regenerating ability of cells thereof to derive detoxication effect and restoration of damaged gingival cells. Cations generated from silver actively assist hormone release and maintain skin health.

Water contained as the balance of the dentifrice composition is purified water preferably containing mineral-free distilled water or pure water in amount of 10 to 50 wt. % relative to total weight of the composition.

The dentifrice composition includes additional components including, for example, solvent, flavor, flavoring agent and sweetening agent. The solvent includes 0.5 to 12 wt. %, preferably 0.5 to 10 wt. % of ethanol. The flavor contains 0.1 to 2 wt. % of at least one or two selected from a group primarily consisting of peppermint oil, herb oil, eucalyptus oil, spearmint oil, orange oil and other composite flavor as food additive. The flavoring agent includes at least one or two selected from 0.05 to 0.3 wt. % of L-menthol and 0.005 to 0.1 wt. % of DL-camphor. The sweetening agent contains 0.1 to 3 wt. % of xylitol and optionally 0.05 to 3 wt. % and, preferably, 0.05 to 0.15 wt. % of sodium saccharine.

Alternatively, the dentifrice composition further includes artificial preservative to prevent teeth from contamination by microorganisms during use of the composition. Such preservative comprises at least one or two selected from 0.01 to 0.2 wt. % of methyl p-hydroxybenzoate and 0.01 to 0.1 wt. % of propyl p-hydroxybenzoate in order to protect teeth from cavity. Preferably, amount of methyl p-hydroxybenzoate ranges from 0.01 to 0.2 wt. %. The dentifrice composition further includes at least one or two of fluorine based compounds widely used as cavity prevention components in trace amount such as 0.02 to 0.22 wt. % of sodium fluoride and 0.2 to 0.76 wt. % of sodium monofluorophosphate.

The dentifrice composition also contains at least one or two selected from gingival disease protection components such as 0.01 to 0.5 wt. % of tocopherol acetate, 0.01 to 0.3 wt. % of allantoin and 0.01 to 0.5 wt. % of allantoin chlorohydroxyaluminum.

Next, the mousse type dentifrice formulation formed by using the dentifrice composition which comprises silver particles as essential ingredient according to the present invention will be more clearly understood by the following description.

In a preferred embodiment of the present invention, a mousse type dentifrice formulation is prepared by introducing the dentifrice composition comprising silver particles as essential ingredient into a foam generating receptacle with constant capacity and drawing the composition in foam form out of the receptacle in order to brush teeth.

The receptacle is preferably an aerosol form of receptacle which is filled with gas. The aerosol form of receptacle has a construction of mixing the dentifrice composition with gas and outputting the mixture by pressing a button of the receptacle or alternative construction consisting of an inner container for receiving the dentifrice composition and an outer container to house the inner container inside while being filled with gas around outside of the inner container.

The gas used in the aerosol form of receptacle includes one or two selected from liquefied natural gas or compressed air. The liquefied natural gas is a mixture of propane gas and butane gas in mixing ratio of 30:70 or 100% butane gas. Amount of the gas filled in the receptacle ranges from 3 to 15 wt. % relative to total amount of the composition introduced in the receptacle.

Meanwhile, the mousse type dentifrice formulation according to the present invention adopts of course a construction of introducing the dentifrice composition in a normal pump type foam generating receptacle then discharging the composition in mousse form outside. For example, such pump type foam generating receptacle consists of: a main body with constant capacity to contain the dentifrice composition; a pump device that is arranged on a fixture cap coupled to output hole of the main body and has an inhalation nozzle which is got into the main body to suck content of the main body and an output nozzle through which the suck dentifrice composition is discharged out of the receptacle. The pump type foam generating receptacle with the above construction is conventionally fabricated in different designs and employed in a wide range of applications including, for example, a container for liquid formulations such as detergent or cosmetics. Therefore, the description of the present invention does not include detailed illustration for the pump type receptacle.

Features of the present invention described above and other advantages will be more clearly understood by the following non-limited examples, which are not intended to restrict the scope of the invention but are instead illustrative embodiments of the present invention.

### ADVANTAGEOUS EFFECTS

As described in detail above, the present invention provides a liquid type dentifrice composition primarily containing silver particles which can efficiently remove bacteria in mouth and protect mouth and teeth from periodontal disease and other mouth diseases, and a mousse type dentifrice formulation comprising the above dentifrice composition with improved aesthetic property and product quality thereof.

### BEST MODE FOR CARRYING OUT THE INVENTION

### Example 1

As listed in Table 1 below, introduced in a mixer are: mouth cleaner ingredients such as 2.5 wt. % of sodium lauryl sulfate, 0.5 wt. % of sodium sarcosine laurate and 3 wt. % of alkyl polyglucoside; wetting agents such as 50 wt. % of sorbitol solution and 3 wt. % of glycerine; sweetening agents such as 0.15 wt. % of sodium saccharine and 0.3 wt. % of xylitol; solvent such as 3 wt. % of ethanol; solubilizing agent such as 0.4 wt. % of hydrogenated polyoxyethylene caster oil; flavor such as 0.6 wt. % of spearmint oil; flavoring agent such as 0.15 wt. % of L-menthol; preservative such as 0.1 wt. % of methyl p-hydroxybenzoate; 36.2 wt. % of purified water; and 0.1 wt. % of silver foil as bactericidal silver particles. The mixture is homogeneously blended in the mixer under vacuum condition, resulting in a dentifrice composition in liquid form for mouthwash.

### Example 2

As listed in Table 1 below, introduced in a mixer are: mouth cleaner ingredients such as 2.5 wt. % of sodium lauryl sulfate and 1 wt. % of sodium sarcosine laurate; wetting agents such as 70 wt. % of sorbitol solution and 3 wt. % of glycerine; sweetening agents such as 0.15 wt. % of sodium saccharine and 0.3 wt. % of xylitol; solvent such as 3 wt. % of ethanol; solubilizing agent such as 0.2 wt. % of hydrogenated polyoxyethylene caster oil; flavor such as 0.9 wt. % of spearmint oil; flavoring agent such as 0.1 wt. % of L-menthol; preservative such as 0.1 wt. % of methyl p-hydroxybenzoate; 18.67 wt. % of purified water; and 0.08 wt. % of silver foil as bactericidal silver particles. The mixture is homogeneously blended in the mixer under vacuum condition, resulting in alternative dentifrice composition in liquid form for mouthwash.

### Example 3

As listed in Table 1 below, introduced in a mixer are: mouth cleaner ingredient such as 2.5 wt. % of sodium lauryl sulfate; wetting agents such as 60 wt. % of sorbitol solution and 3 wt. % of glycerine; sweetening agents such as 0.15 wt. % of sodium saccharine and 0.3 wt. % of xylitol; solvent such as 3 wt. % of ethanol; solubilizing agent such as 0.4 wt. % of hydrogenated polyoxyethylene caster oil; flavor such as 0.9 wt. % of spearmint oil; flavoring agent such as 0.1 wt. % of L-menthol; preservative such as 0.1 wt. % of methyl p-hydroxybenzoate; 29.54 wt. % of purified water; and 0.01 wt. % of silver foil as bactericidal silver particles. The mixture is homogeneously blended in the mixer under vacuum condition, resulting in alternative dentifrice composition in liquid form for mouthwash.

### Comparative Example 1

As listed in Table 1 below, introduced in a mixer are: wetting agents such as 8 wt. % of sorbitol solution and 3 wt. % of glycerine; sweetening agents such as 0.15 wt. % of sodium saccharine and 0.3 wt. % of xylitol; solvent such as 3 wt. % of ethanol; solubilizing agent such as 0.4 wt. % of hydrogenated polyoxyethylene caster oil; flavor such as 0.4 wt. % of spearmint oil; flavoring agent such as 0.1 wt. % of L-menthol; preservative such as 0.1 wt. % of methyl p′-hydroxybenzoate; and 84.55 wt. % of purified water. The mixture is homogeneously blended in the mixer under vacuum condition, resulting in a different dentifrice composition in liquid form for mouthwash.

TABLE 1

| Kinds of | | | | | Comparative |
|---|---|---|---|---|---|
| ingredients | Name of ingredients | Ex. 1 | Ex. 2 | Ex. 3 | Ex. 1 |
| Cleaner | Sodium lauryl sulfate | 2.5 wt. % | 2.5 wt. % | 2.5 wt. % | — |
| ingreidnets | Sodium sarcosine laurate | 0.5 wt. % | 1 wt. % | — | — |
| | Alkyl polyglucoside | 3 wt. % | — | — | — |
| Wetting | Sorbitol solution | 50 wt. % | 70 wt. % | 60 wt % | 8 wt. %. |
| agent | Glycerine | 3 wt. % | 3 wt. % | 3 wt. % | 3 wt. % |
| Sweetening | Sodium saccharide | 0.15 wt. % | 0.15 wt. % | 0.15 wt. % | 0.15 wt. % |
| agent | Xylitol | 0.3 wt. % | 0.3 wt. % | 0.3 wt. % | 0.3 wt. % |
| Solvent | Ethanol | 3 wt. % | 3 wt. % | 3 wt. % | 3 wt. % |
| Solubilizing | Hydrogenated Polyoxyethylene | 0.4 wt. % | 0.2 wt. % | 0.4 wt. % | 0.4 wt. % |
| agent | caster oil | | | | |
| Flavor | Spearmint oil | 0.6 wt. % | 0.9 wt. % | 0.9 wt. % | 0.4 wt. % |
| Flavoring | L-menthol | 0.15 wt. % | 0.1 wt. % | 0.1 wt. % | 0.1 wt. % |
| agent | | | | | |
| Preservative | Methyl p-hydroxybenzoate | 0.1 wt. % | 0.1 wt. % | 0.1 wt. % | 0.1 wt. % |
| Purified | Purified water | 36.2 wt. % | 18.67 wt. % | 29.54 wt. % | 84.55 wt. % |
| water | | | | | |
| Bactericidal | Silver foil | 0.1 wt. % | 0.08 wt. % | 0.01 wt. % | — |
| agent | | | | | |

### Experimental Example

The experimental example described below is to determine whether each of the prepared dentifrice compositions according to Examples 1 to 3 and Comparative Example 1 is drawn out in foam state from a receptacle when used to prevent the composition from flowing down, so that the composition can maximize performance of tooth-brushing. In order to compare all of the dentifrice compositions with one another and apply an objective evaluation to the compared results, the compositions were subjected to measurement of foam generation ability and foam duration time by using a measuring flask and according to free falling test as an indirect method.

The experimental method is practically implemented by steps of: free flowing test solution containing any one of the prepared dentifrice composition at 25° C. from a constant height into a measuring flask with capacity of about 100 ml in a desired period of time; measuring height of foam generated in the measuring flask; and determining time passed while original height of the foam is reduced to a point with 30% of the original height, by which foam duration time was estimated. The determined result is shown in Table 2 below:

TABLE 2

| | | | | Compar- |
|---|---|---|---|---|
| Sort | Ex. 1 | Ex. 2 | Ex. 3 | ative ex. 1 |
| Foam | 13 cm | 10 cm | 7 cm | 3 cm |
| height | | | | |
| Foam | 6 min. 10 sec. | 5 min. 20 sec. | 3 min. 10 sec. | 55 sec. |
| duration | | | | |
| time | | | | |

Following to the above illustrated experimental example and as shown in Table 2, it was clearly demonstrated that each of the present dentifrice compositions from Examples 1 to 3 has foam height at least 2 to 4 times higher than that of the dentifrice composition from Comparative Example 1 as a control and foam duration time at least 3 to 6 times longer than that of the control. Consequently, when the liquid type dentifrice composition primarily containing silver particles according to the present invention is used to brush teeth, the present composition shows excellent foam generation ability and long foam duration time sufficient to prevent injected liquid dentifrice from easily flowing down from a toothbrush, resulting in effective and convenient tooth-brushing function.

While the present invention has been described with reference to the above examples and experimental example, which are not intended to restrict the scope of the invention but are instead illustrative examples of the present invention. Accordingly, it will be obvious to those skilled in the art that various modifications and variations may be made therein without departing from the scope of the present invention as defined by the appended claims.

### INDUSTRIAL APPLICABILITY

As described in detail above, the liquid type dentifrice composition primarily containing silver particles and the mousse type dentifrice formulation according to the present invention have higher foam generation ability and longer foam duration time sufficient to prevent the liquid dentifrice from easily flowing down from a toothbrush, resulting in effective and convenient tooth-brushing function as well as superior mouth and tooth cleaning effect by cleaner ingredients in the generated foam.

In addition, the composition can exhibit bactericidal action and protect mouth and teeth from periodontal disease and other mouth diseases because of original properties of silver ingredients in the dentifrice composition, which comprises natural silver particles instead of chemical ingredients. Further, the mousse type dentifrice formulation prepared by using the dentifrice composition according to the present invention improves aesthetic property and, in turn, product quality thereof.

What is claimed is:

1. A liquid type dentifrice composition primarily containing silver particles useful for mouthwash, which comprises: at least one surfactant selected from a group consisting of 0.5 to 3% by weight of sodium lauryl sulfate, 0.05 to 1% by weight of sodium sarcosine laurate, 0.5 to 5% by weight of alkyl polyglucoside and 0.5 to 3% by weight of sucrose fatty acid ester; at least one wetting agent selected from a group consisting of 30 to 80% by weight of sorbitol solution and 0.5 to 5% by weight of glycerine; at least one solubilizing agent selected from a group consisting of 0.1 to 0.1% by weight of hydrogenated polyoxyethylene caster oil and 0.1 to 1% by weight of polyoxyethylene sorbitan monolaurate effective to solubilize flavor and active ingredients; 0.01 to 10% by weight of silver particles with bactericidal action; and the balance of purified water relative to total weight of the composition.

2. The dentifrice composition according to claim 1, wherein the silver particle moiety in the composition comprises 0.01 to 0.1% by weight of silver foil having particle diameter of 1 to 200 μm.

3. The dentifrice composition according to claim 1, wherein the dentifrice composition further includes: solvent containing 1 to 12% by weight of ethanol; 0.5 to 2% by weight of at least one flavor ingredient selected from a group consisting of peppermint oil, herb oil, eucalyptus oil, spearmint oil, orange oil and other composite flavor component as food additive; at least one flavoring agent selected from a group consisting of 0.05 to 0.3% by weight of L-menthol and 0.005 to 0.1% by weight of DL-camphor; and 0.05 to 0.15% by weight of sodium saccharin.

4. The dentifrice composition according to claim 1, wherein the dentifrice composition further includes active ingredient for protecting gingival disease comprising at least one selected from a group consisting of 0.01 to 0.5% by weight of tocopherol acetate, 0.01 to 0.3% by weight of allantoin and 0.01 to 0.5% by weight of allantoin chlorohydroxyaluminum.

5. The dentifrice composition according to claim 1, wherein the dentifrice composition further includes active ingredient for protecting cavity comprising at least one selected from 0.02 to 0.22% by weight of sodium fluoride and 0.2 to 0.76% by weight of sodium monofluorophosphate, as well as preservative containing 0.01 to 0.2% by weight of methyl p-hydroxybenzoate.

6. A mousse type dentifrice formulation comprising the liquid dentifrice composition as defined in any one of claims 1 to 5, which is prepared by steps of: introducing the dentifrice composition comprising silver particles as essential ingredient into a foam generating receptacle with constant capacity; and drawing the composition in foam form through outlet of the receptacle in order to brush teeth.

7. The mousse type dentifrice formulation according to claim 6, wherein the receptacle is a pump type foam generating receptacle consisting of: a main body with constant capacity to contain the dentifrice composition; a pump device that is arranged on a fixture cap coupled to output hole of the main body and has an inhalation nozzle which is got into the main body to suck content of the main body and an output nozzle through which the suck dentifrice composition is discharged out of the receptacle.

8. The mousse type dentifrice formulation according to claim 6, wherein the receptacle is an aerosol form of receptacle with a construction of mixing the dentifrice composition with gas and outputting the mixture by pressing a button of the receptacle, or alternative construction consisting of an inner container for receiving the dentifrice composition and an outer container to house the inner container inside while being filled with gas around outside of the inner container.

9. The mousse type dentifrice formulation according to claim 8, wherein the gas filled in the aerosol form of receptacle is a mixture of propane gas and butane gas in mixing ratio of 30:70, liquefied petroleum gas with 100% butane gas or compressed air.
